(19) United States
(12) Patent Application Publication
(10) Pub. No.: US 2004/0248127 A1
(43) Pub. Date: Dec. 9, 2004

(54) Method for separating macromolecules having been chemically modified in a reversible manner

(76) Inventors: Kai H. Te Kaat, Frankfurt (DE); Christian Hamon, Frankfurt (DE); Richard Joubert, Frankfurt (DE); Thomas Neumann, Frankfurt (DE); Wolfgang Schwier, Kelkheim (DE)

Correspondence Address: CONNOLLY BOVE LODGE & HUTZ, LLP, P O BOX 2207, WILMINGTON, DE, 19899, US

(21) Appl. No.: 10/487,535

(22) PCT Filed: Sep. 3, 2002
(86) PCT No.: PCT/EP02/09808

(30) Foreign Application Priority Data
Sep. 4, 2001 (DE) 101 43 346.8

(51) Int. Cl. C12Q 1/68; G01N 33/53

(52) U.S. Cl. 435/6; 435/7.1

(57) ABSTRACT

The present invention relates to a method for fractionating a sample of macromolecules, in particular for electrophoretically separating proteins, characterized in that at least one macromolecule in the sample is modified, before carrying out the separation method, with at least one group which can be partially or completely eliminated once again under mild conditions.

Method for separating macromolecules having been chemically modified in a reversible manner

The present invention relates to a method for fractionating a sample of macromolecules, characterized in that, before the separation method is carried out, at least one macromolecule in the sample is modified with at least one group which can be partially or completely eliminated once again under mild conditions.

Electrophoretic and chromatographic separation methods make use of the physical or biochemical properties, such as electric charge, size or molecular weight, of the compounds to be separated. In these methods, problems of separation arise in the case of compounds which possess similar physical or biochemical properties. For example, the separation of hydrophobic proteins, in particular membrane proteins, which cannot be separated using the standard separation methods, such as two dimensional gel electrophoresis, causes particular problems in this connection.

In two dimensional gel electrophoresis, a first step in the form of an isoelectric focusing, in which the species in the sample are separated in accordance with their pI values, is as a rule followed by a separation step which is perpendicular to the first step and is in the form of an SDS gel electrophoresis in which the species in the sample are primarily separated in accordance with their molecular weights.

A fundamental problem in regard to the isoelectric focusing of membrane proteins or hydrophobic proteins in general is the fact that, in this method, it is only possible to use uncharged, that is neutral or zwitterionic, detergents and not possible to use ionic detergents, such as SDS, which are most well-suited for solubilizing membrane proteins.

The reasons for the problems in regard to separating membrane proteins by means of isoelectric focusing are therefore, in particular, inadequate solubilization of the membrane proteins in a sample, adsorption of the hydrophobic proteins to the polyacrylamide gel matrix and problems with the solubility of the membrane proteins at the isoelectric point.

Consequently, two dimensional gel electrophoresis, which can conventionally be used for characterizing a protein mixture, is completely unsuitable for membrane proteins, in particular.

The object of the present invention was, therefore, to make available a method for separating macromolecules which, because of their biochemical or physical properties, cannot be separated, or can only be separated with difficulty, when using the conventional separation methods.

The object of the present invention was, in particular, to make available a multidimensional method for separating hydrophobic proteins, especially membrane proteins, with this method being better-suited for separating hydrophobic proteins than the previously described, and conventionally employed, separation methods.

According to the invention, this object is achieved by means of a method in which, before the separation method is carried out, the macromolecules in the sample are incubated with at least one group which is able to bind covalently to at least one of the macromolecules in the sample and which, after binding to the macromolecule, can be completely or partially removed once again under mild conditions such that, after the incubation, at least one macromolecule in the sample has been modified with at least one group and, after the separation method has been carried out, the modifying group can be partially or completely removed once again such that, where appropriate, the unmodified starting compounds, or the starting compounds which are only provided with a barcode, can be obtained once again. It is then possible, where appropriate, to carry out further separation steps using the macromolecules which have been demodified in this way.

The prior art (WO 99/19514) has thus far only described methods in which, before a separation method is carried out, either macromolecules are covalently modified irreversibly, i.e. such that it is not possible to eliminate the modifying group under mild conditions, or in which completely nonspecific labeling with intercalating dyes, which do not bind covalently, takes place. Neither method is suitable for achieving the object in accordance with the invention.

The present invention consequently relates to a method for fractionating a sample of macromolecules, which method is characterized in that, before the separation method is carried out, at least one macromolecule in the sample is chemically modified reversibly with at least one group, with reversible chemical modification meaning that the group which has been inserted can be partially or completely eliminated from the macromolecule once again under mild conditions.

Selectively and specifically modifying macromolecules in the sample in this way selectively and specifically alters the physical and/or biochemical properties of the macromolecules, such as the molecular weight, the polarity or the electric charge, or result in the introduction of a label, such as a magnetic or radioactive label, such that it is then possible to separate the macromolecules using appropriate methods which are based on the abovementioned properties.

In this connection, the method is particularly suitable for separating macromolecules which possess identical or very similar properties, in particular, for example, identical or similar molecular weights, but which differ from each other in the number of their functional, chemically modifiable groups. For example, it is possible, in this way, by means of reacting with modifying groups, to increase the molecular masses of the individual macromolecules to differing extents, in dependence on the functional groups, such that it then becomes possible to separate these macromolecules.

According to the invention, the macromolecules are, in this connection, for example, carbohydrates, nucleic acids or proteins, in particular hydrophobic proteins, especially membrane proteins, or compounds or complexes which comprise at least one of the aforementioned compounds. The compound can, for example, be a glycoprotein and the complex can, for example, be a membrane protein which has been solubilized using detergents. According to the invention, these macromolecules can also be modified in a manner which is known to the skilled person.

The macromolecule sample is, correspondingly, a sample which comprises at least two macromolecules which are selected from at least one of the previously mentioned groups, with the sample preferably being a sample which comprises at least two macromolecules which, because of their biochemical or physical properties, or for technical reasons, cannot be separated, or can only be separated with difficulty, in particular insufficiently, in a separation method.

In a preferred embodiment, the macromolecule sample completely or partially comprises a naturally occurring ensemble, such as the genome or the proteome of a cell and/or of an organelle, especially the proteome of a cell membrane or of an organelle membrane, with “partially” preferably meaning that the sample comprises at least 5, particularly preferably at least 10, macromolecules, especially at least 20 macromolecules, of such an ensemble.

While, according to the invention, the sample comprises at least one macromolecule which can be modified with a modifying group, preference is given to it being possible to modify several, or all, of the macromolecules in the sample with at least one, preferably several, modifying groups.

According to the invention, the modification of the at least one macromolecule with the at least one modifying group is rendered possible by the macromolecule and the modifying group in each case comprising a functional group, which functional groups are able to form a linkage, preferably a covalent linkage, with each other. For example, the macromolecule comprises at least one nucleophilic group and the modifying group comprises at least one electrophilic group, or vice versa. The at least one functional group in the macromolecule is also termed “reactive group” below in order to distinguish it from the at least one functional group belonging to the modifying group.

Correspondingly, the modifying group can, for example, and in dependence on the reactive group possessed by the macromolecule, comprise at least one of the following functional groups: a hydrazide or amino group, for reacting with an aldehyde group, a thiol group, a haloacetyl derivative, e.g. RCOCH₂I, a male-imide group or a vinylsulfone group, for reacting with a thiol group, or an active ester, e.g. NHS, an aldehyde group, an isthiocyanate, an isocyanate, an acylazide derivative, a sulfonyl chloride, an activated carbonate derivative, an imido ester or an acid anhydride, for reacting with an amino group.

The at least one reactive group, which the macromolecule comprises, is preferably a primary, secondary or tertiary amino group and/or a thiol group. In a particular embodiment, these groups can be present in amino acids or amino acid residues. The reactive groups can either be present naturally in the macromolecule or have been introduced into the macromolecule in a manner known to the skilled person. Preferably, several reactive groups are present in a macromolecule which is to be modified.

In a preferred embodiment, the reactive groups are the amino groups of the lysine residues and the N-terminal amino group and/or the thiol groups of the cysteine residues present in naturally occurring proteins.

In another preferred embodiment, the reactive groups are keto or aldehyde groups, for example aldehyde groups which are present in the sugar component or glycoproteins.

The macromolecules can also contain several identical or different reactive groups which may be able, for example, to react selectively with different modifying groups. It is likewise possible for the modifying group to contain several identical or different functional groups. Macromolecules which possess several reactive groups can be modified simultaneously or successively with several modifying groups.

The modifying group is distinguished by the fact that it is either able to form a bond, which can be cleaved once again under mild conditions, with at least one macromolecule in the sample or is able to enter into a stable bond, which cannot be cleaved under mild conditions, with the macromolecule, but with the modifying group then comprising a bond which can be cleaved under mild conditions, such that, when this bond is cleaved, part, preferably the major part, of the modifying group can be removed once again. This ensures that, depending on the intended purpose, the modifying group can, after the separation method has been carried out, be removed again, either completely or partially, thereby making it possible to once again obtain either the completely demodified, or the extensively demodified, macromolecule.

In the first case, that is when the modifying group can once again be removed completely, the reactive group and the functional group are preferably in each case a thiol group. The modifying group can then be introduced under mild oxidizing conditions and removed once again under mild reducing conditions.

In the second case, the reactive group and the functional group are in each case one of the previously mentioned groups which are able to form, with each other, a stable covalent bond which cannot be readily cleaved under mild chemical conditions, such as, for example, a C—C bond, an amide bond, especially a peptide bond, or an ester bond, or another stable bond, especially a covalent bond which is known to the skilled person. However, in this case, the modifying group comprises a bond which can be cleaved under mild reaction conditions.

According to the invention, a bond which can be cleaved under mild conditions means, for example, a bond which can be cleaved under mildly reductive or mildly oxidative conditions, or a bond which can be cleaved with a slight increase or decrease in the pH and/or the redox potential, or, for example, a bond which can be cleaved photolytically. In general, this furthermore means conditions under which cleavage of the covalent bonds which are normally met with, as are met with in the macromolecules, such as proteins, which are present in the sample before carrying out the modification, does not take place; that is, the bond can be cleaved under reaction conditions under which at least the primary structure of the macromolecules is not altered. In another preferred embodiment, the cleavage can also be effected while retaining the secondary or tertiary structure of the macromolecules.

In particular, the group which can be cleaved under mild conditions can, in this connection, for example, be a disulfide bridge, which can be cleaved by making the medium reductive, or, for example, be a vicinal cis-diol group, which can be cleaved with sodium periodate (NaIO₄) or lead tetraacetate (Pb(OAc)₄), or, for example, be a photolytically cleavable group, such as, for example, a 1-(2-nitrophenyl)ethyl ester, which can be cleaved with UV radiation at 300-360 nm, or be another photolytically cleavable group as can be found, for example, in one of the following publications: Proc. Natl. Acad. Sci. (1995) 92: 7590-4: Photocleavable biotin derivatives: a versatile approach for the isolation of biomolecules; J. Org. Chem. (1997) 62: 2370-80: Model studies for new o-nitrobenzyl photolabile linkers: substituent effects on the rates of photochemical cleavage.

The bond which can be cleaved under mild chemical conditions can be located in any region of the modifying group. However, in a preferred embodiment, it is located in the vicinity of the stable bond which has been formed between the macromolecule and the modifying group such that, when this bond is cleaved, most of the modifying group is removed once again, with preference being given, once the modifying group or modifying groups has/have been removed in this way, to the residue of this/these group(s) remaining on the macromolecule amounting to not more than 10%, particularly preferably not more than 5%, in particular not more than 2%, of the molecular weight of the demodified macromolecule, such that the original molecular weight of the macromolecule is to a large extent restored once again.

In one particular embodiment, the residue of the modifying group(s) which remains on the macromolecule in this connection is a barcode. In this connection, the barcode is, preferably, a group which can be used for detection for the purpose of in-gel hybridization with, for example, a radioactively labeled oligonucleotide and/or a group which makes it possible to distinguish treated macromolecules from control macromolecules and, where appropriate, to separate these macromolecules on a gel and detect them separately.

In this sense, therefore, any group which makes it possible to differentiate untreated and control molecules is suitable for use as a barcode. However, according to the invention, the barcode preferably comprises at least one oligonucleotide or polynucleotide, in particular one RNA or DNA, or one so-called peptide nucleic acid (PNA), with it being possible for these compounds to be present in either single-stranded or double-stranded form. In another preferred embodiment, the nucleic acids are labeled with at least one aromatic group, for example with a heterocyclic group.

When modifying the macromolecules in the sample, preference is given to selecting the conditions such that the species in the sample are modified homogeneously. In this connection, homogenous means that the macromolecules in the sample are labeled to differing degrees depending on the number of reactive groups which a single macromolecule contains.

In the connection, the conditions can be selected such that, for example, all the reactive groups in a macromolecule are reacted with the modifying group or else only a particular percentage of the reactive groups in a macromolecule are reacted with the modifying group, for example as a result of using a relative low concentration of the modifying groups.

When modifying native macromolecules, for example, the reaction conditions can also be selected such that only particular reactive groups are reacted with the modifying group, for example those groups which, on account of their nature, exhibit higher reactivity than do other groups, or those groups which, on account of the tertiary structure of the macromolecule, are more readily accessible than are other groups, for example because they are exposed on the outside.

Such a selective labeling can, for example, be achieved by selecting the reaction conditions, during the incubation with the at least one modifying group, such that it is possible to distinguish between the reactivity of the different reactive and/or functional groups or, in the latter case, by the tertiary structure of the macromolecules being at least partially preserved, resulting in only the reactive groups which are exposed on the outside, and not those which are facing inwards, being accessible to the modification.

According to the invention, the separation method comprises at least one electrophoretic or chromatographic separation step. In this connection, the sample is preferably prepared, before being loaded on the gel, in a suitable manner as known to the skilled person for carrying out appropriate separation methods, for example polyacrylamide gel electrophoresis. According to the invention, particular preference is given to using polyacrylamide gels which have a high separation efficiency for carrying out the polyacrylamide gel electrophoresis. According to the invention, the separation method can be used for both analytical and preparative purposes.

In a preferred embodiment, the separation method is a multidimensional, especially two dimensional, separation method, with, in a particularly preferred embodiment, the separation method being carried out, in this context, such that the macromolecule sample is incubated with at least one modifying group and at least one separation step is then carried out with the sample which has been treated in this way, and which contains at least one modified macromolecule, after which the modifying groups are completely or partially removed and, subsequently, at least one separation step is likewise carried out with the macromolecules which have been demodified in this way.

Alternatively or additionally, it is naturally also possible to carry out one or more separation steps with the untreated macromolecules even before the macromolecules have been modified.

The separation steps in this connection can, independently of each other, be chromatographic or electrophoretic methods. In a particularly preferred embodiment, polyacrylamide gel electrophoresis (PAGE) is used for separating the macromolecules.

In this regard, the particular advantage ensues, especially when separating hydrophobic proteins, in particular membrane proteins, that it is possible, instead of the isoelectric focusing, which is usually employed as the first separation step in two dimensional gel-electrophoretic methods, to carry out a separation step with the proteins in which it is also possible to use ionic detergents, which are particularly suitable for solubilizing the membrane proteins.

According to the invention, the first separation step therefore preferably consists, particularly in this case, in using SDS-PAGE to electrophoretically separate the proteins which have been chemically modified reversibly. The second step then takes place, preferably at about right angles to the first direction of separation, after the modifying groups have been completely or partially removed, with this step being the electrophoretic separation of the demodified proteins, which is likewise effected using SDS-PAGE.

A particular advantage of the 2D gel method according to the invention as compared with the known 2D methods is that, while ionic detergents can already be used in the first separation step, they can also even be used, in particular, in preparing the sample. This makes it possible, in particular, to separate even strongly hydrophobic proteins, especially membrane proteins, which is not possible using the conventional techniques of 2D gel electrophoresis.

### FIGURES

FIG. 1 shows a diagram of a preferred embodiment of the method. In this embodiment, the modified macromolecules are used to carry out a separation step in the first dimension and, after the modifying group has been removed, a separation step then takes place in the second dimension, preferably at right angles to the first direction of separation.

FIG. 2 shows a diagram of a modified macromolecule. The oval circle labeled P identifies the macromolecule, for example a protein. The residue diagrammatically depicts one particular embodiment of a modifying group. In this connection, the modifying group comprises a variable region (tractor) and a constant region (barcode), which regions are separated from each other by a group which can be cleaved under mild conditions. In this embodiment, the modifying group can be removed under mild conditions while a barcode remains. The barcode makes it possible, for example, to differentiate modified macromolecules and unmodified macromolecules.

FIG. 3 depicts the correlation between the native molecular weight and the number of lysines in the case of known membrane proteins derived from the yeast Saccharomyces cerevisiae, while FIG. 4 depicts a corresponding correlation between native molecular weight and the number of cysteines. As can be seen, membrane proteins having similar molecular weights possess differing numbers of cysteine and/or lysine residues such that modifying these reactive groups alters the molecular weights of the macromolecules to differing extents.

FIG. 5 diagrammatically depicts the performance of the reaction, as explained in implementation Example 1. In this connection, R-NH₂ represents a macromolecule possessing a reactive group. This macromolecule is reacted with the reagent NHS-SS-biotin resulting in the formation of the compound which is depicted. The disulfide group can then be cleaved under mild reaction conditions, resulting in the removal of the greater part of the modifying group.

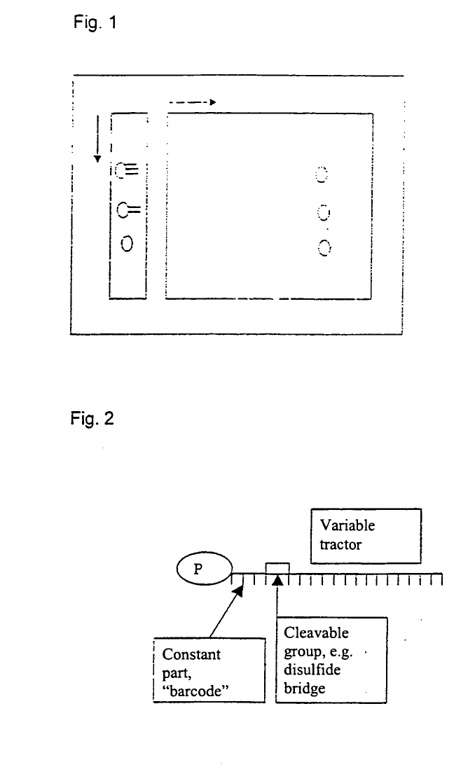

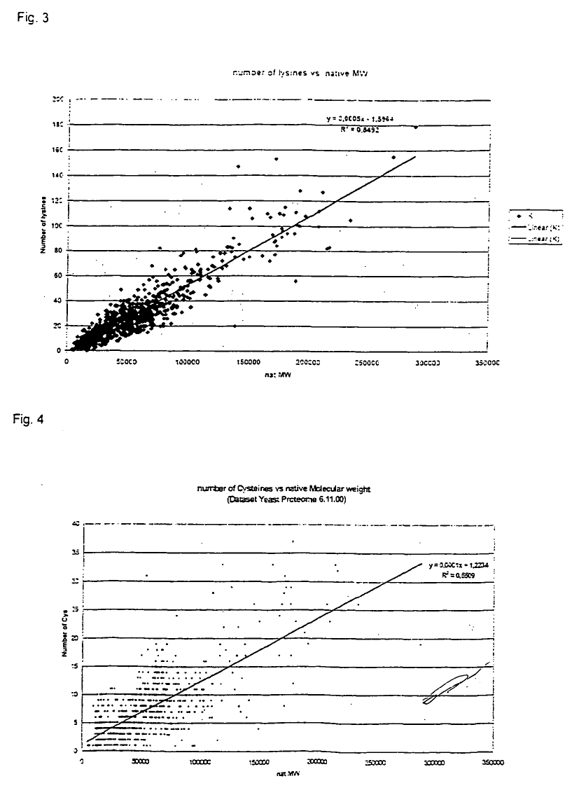

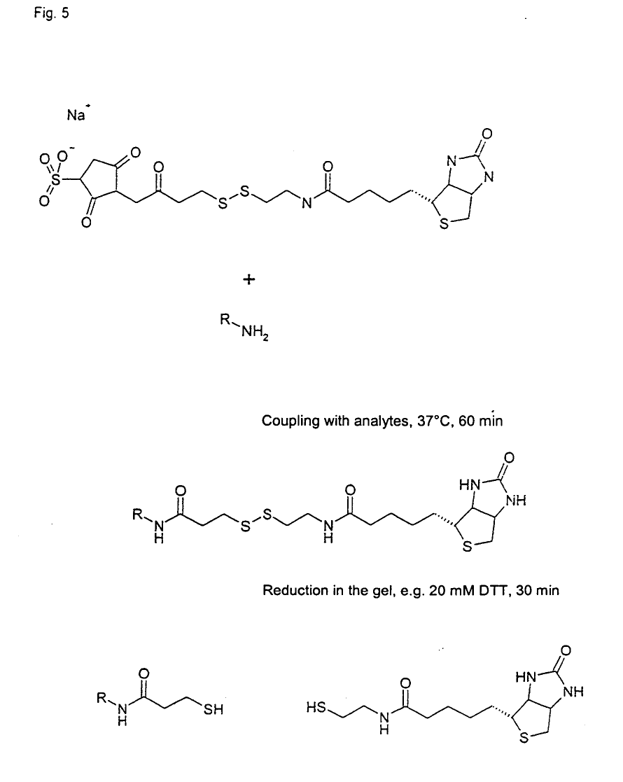

### IMPLEMENTATION EXAMPLES

Model proteins (soluble membrane proteins and integral membrane proteins) were denatured with 1% SDS solution and modified with NHS-SS-biotin (selective for primary amines) at their aminoterminal NH₂ and lysine side chains. The reagent leads to an increase in mass of 391 Da/functional group. Eliminating the biotin group by reducing with DTT led to what was approximately the native molecular weight (loss of 303 Da/functional group) In model experiments, a protein mixture consisting of four different proteins was labeled with NHS-SS-biotin or, respectively, with purified Rhodovulum sulfidophilum cytochrome bsl complex.

Reaction conditions (final concentrations): Protein, in each case 0.1 mg/ml; 125 mM HEPES, from pH 8.5 to 9.0; 1% (w/v) SDS, NHS-SS-biotin, up to 2 mM final concentration, DMSO, up to 10% final concentration. The proteins were dissolved in HEPES/SDS buffer at 37° C. and the reaction was started by adding biotinylating reagent (dissolved in DMSO) up to a concentration of 1 mM. After approx. 30 min, the second half of the biotinylating reagent was added, to give the final concentration of 2 mM, and the mixture was incubated once again at 37° C. for 30 min. The reaction mixture was analyzed by means of SDS-PAGE. In this way, it was possible to achieve an increase in the molecular weight (in the region of 10%), which increase was reversible by reducing with DTT.

The following table lists the Rf values of the model proteins before and after being modified with the biotin linker. Reduction with DTT resulted in what were approximately the native Rf values.

| | | | Rf, | Rf, |
|---|---|---|---|---|
| | Molecular | Number of | native | modified |
| Protein | weight | amino groups | protein | protein |
| BSA | 66 kDa | 59 K + N-term | 0.272 | 0.238 |
| trypsin | 21 kDa | 12 K + N-term | 0.776 | 0.646 |
| inhibitor | | | | |
| lysozyme | 14 kDa | 6 K + N-term | 0.884 | 0.871 |
| FtsY (e. coli, | 54 kDa | 33 K + N-term | 0.122 | 0.136 |
| P10121) | (app. MW | | | |
| | 97 kDa) | | | |
| D48-FtsY | 49 kDa | 27 K + N-term | 0.231 | 0.204 |
| (E. coli) | (app MW 60 | | | |
| | kDa) | | | |
| cytochrome | | By homology: | | |
| bc1 | 50 kDa | 11 K + N-term | 0.737 | 0.635 |
| cytochrome b | 34 kDa | 14 K + N-term | 0.599 | 0.511 |
| cytochrome c1 | (diffuse | 8 K + N-term | 0.438 | 0.365 |
| | band) | | | |
| Rieske | 25 kDa | | | |

The Rf values are the distances migrated by the proteins divided by the distance migrated by bromophenol blue. Proteins possessing high Rf values run close to the front. The purified cytochrome bc1 complex was kindly provided by Prof. Irmgard Sinning, BZH Heidelberg. The sequence of the complex employed is not known; for this reason, the composition of the bc1 complex derived from the closely related Rb. Capsulatus was used in this present case. All the subunits of the complex are integral membrane proteins possessing 1 transmembrane helix (Cyt cl and Rieske) or 8 transmembrane helices (Cytochrome b). All the other model proteins are soluble proteins.

1. A method for fractionating a sample of macromolecules, characterized in that, prior to the separation method, at least one macromolecule in the sample is covalently linked to at least one modifying group which can be partially or completely removed under mild conditions.

2. The method as claimed in claim 1, characterized in that the at least one modifying group is linked to the at least one macromolecule by means of reaction with at least one reactive group in the macromolecule.

3. The method as claimed in claim 2, characterized in that the reactive group is a primary, secondary or tertiary amino group, an SH group or an aldehyde group.

4. The method as claimed in claim 2, characterized in that the reactive group is part of an amino acid residue or sugar residue.

5. The method as claimed in claim 1, characterized in that the at least one macromolecule is a protein, a sugar or a nucleic acid or in that this macromolecule comprises at least one of said compounds, with it also being possible for the macromolecule to be modified.

6. The method as claimed in claim 5, characterized in that the macromolecule comprises at least one hydrophobic protein.

7. The method as claimed in claim 6, characterized in that the hydrophobic protein is a membrane protein.

8. The method as claimed in claim 1, characterized in that the macromolecule sample completely or partially comprises the proteome of a cell, of an organelle, of a cell membrane or of an organelle membrane.

9. The method as claimed in claim 1, characterized in that the modifying group comprises a DNA, an RNA and/or a PNA.

10. The method as claimed in claim 1, characterized in that the modifying group comprises at least one functional group selected from the group consisting of thiol group, acyl halide, hydrazide, amino group, haloacetyl derivative, maleimide, vinylsulfone, active ester, aldehyde, isothiocyanate, isocyanate, acylazide, sulfonyl chloride, activated carbonate, imido ester and acid anhydride.

11. The method as claimed in claim 1, characterized in that a bond, which can be cleaved under mild conditions, is formed between the macromolecule and the modifying group.

12. The method as claimed in claim 1, characterized in that a stable chemical bond is formed between the macromolecule and the modifying group and the modifying group comprises at least one bond which can be cleaved under mild conditions.

13. The method as claimed in claim 11, characterized in that the bond which can be cleaved under mild conditions is a disulfide bridge, a photolytically cleavable group or a vicinal diol group.

14. The method as claimed in claim 1, characterized in that the separation method is a chromatographic or electrophoretic method.

15. The method as claimed in claim 14, characterized in that the method is polyacrylamide gel electrophoresis.

16. The method as claimed in claim 1, characterized in that it is a multidimensional method.

17. The method as claimed in claim 1, which comprises the following steps:
a) a macromolecule sample is reacted with at least one modifying group,
b) at least one separation step is carried out using the sample according to (a),
c) the at least one modifying group is entirely or partially eliminated from the modified macromolecules,
d) at least one further separation step is carried out using the sample according to (c).

18. The method as claimed in claim 17, characterized in that the at least one separation step according to (b) and according to (d) is SDS polyacrylamide gel electrophoresis.

19. The method as claimed in claim 17, characterized in that the modifying group according to (c) is eliminated under mild conditions.

20. The method as claimed in claim 19, characterized in that the modifying group is eliminated reductively, oxidatively or photolytically.

21. The method as claimed in claim 19, characterized in that a barcode remains on the macromolecule after the modifying group has been eliminated.

22. The method as claimed in claim 21, characterized in that the barcode comprises at least one nucleic acid or PNA either of which may also be modified.

23. The method as claimed in claim 17, characterized in that the direction of separation in step (d) is different from that in step (b) with the separation in step (d) taking place at about right angles to the separation in step (b).

24. The method as claimed in claim 12, characterized in that the bond which can be cleaved under mild conditions is a disulfide bridge, a photolytically cleavable group or a vicinal diol group.
